(19) United States
(12) Patent Application Publication
(10) Pub. No.: US 2025/0064689 A1
(43) Pub. Date: Feb. 27, 2025

(54) COMPOSITION FOR OCCLUDING DENTINAL TUBULES AND ORAL PROUDUCT COMPRISING THE SAME

(71) Applicant: SEOUL NATIONAL UNIVERSITY R&DB FOUNDATION, Seoul (KR)

(72) Inventor: Deog Gyu SEO, Seoul (KR)

(21) Appl. No.: 18/652,109

(22) Filed: May 1, 2024

(30) Foreign Application Priority Data
Aug. 23, 2023 (KR) 10-2023-0110218

(51) Int. Cl. A61K 6/853; A61K 6/17; A61K 6/74

(52) U.S. Cl. CPC A61K 6/853; A61K 6/74; A61K 6/17

(57) ABSTRACT

The present disclosure relates to a composition for occluding dentinal tubules and an oral product including the same, and more specifically, to a composition that has excellent dentinal tubule-occluding ability and is capable of preventing or alleviating dentin hypersensitivity.

### BACKGROUND

### 1. Technical Field

The present disclosure relates to a composition for occluding dentinal tubules and an oral product comprising the same, and specifically, to a composition and oral product capable of preventing or alleviating dentin hypersensitivity by sealing the inside of dentinal tubules.

The present application claims priority to Korean Patent Application No. 10-2023-0110218 filed in the Republic of Korea on Aug. 23, 2023, the disclosure the disclosure of which is incorporated herein by reference.

### 2. Related Art

A tooth is composed of enamel, which is the outermost layer, dentin, which is below the enamel, and pulp, which is the innermost layer and is surrounded by the dentin. In the dentin, there are a myriad of microscopic tubes called dentinal tubules, which extend from the pulp to the dentin-enamel junction.

The inside of these dentinal tubules is usually filled with fluid, and the diameter of the dentinal tubules increases from the outer dentin toward the pulp. The diameter of the dentinal tubules is approximately 0.8 μm to 2.2 μm.

Dentin hypersensitivity refers to pain that occurs when various stimuli are applied to exposed dentin without lesions in the pulp, and is also commonly called sensitive tooth pain.

Stimuli applied to exposed dentin are caused by temperature changes, chemical changes, and various mechanical stimuli in the oral cavity, and the mechanisms by which such temperature stimuli, chemical stimuli, and mechanical stimuli cause pain have been explained by various theories. According to the most likely hypothesis, it is explained that the changes in volume or pressure caused by stimuli applied to the exposed dentin surface lead to fluid movement into open dentinal tubules, causing pain. Dentin exposure can occur in various situations, and for example, it can be caused by loss of the crown due to incorrect tooth-brushing habits or eating habits, periodontal disease, tooth root exposure due to periodontal treatment, side effects from tooth restoration treatment or whitening treatment, or physiological phenomena due to aging rather than a special disease.

Dentin hypersensitivity symptoms can be alleviated by occluding the dentinal tubules to reduce the movement of fluid in the dentinal tubules or dentin permeability.

Materials for occluding dentinal tubules include materials containing ions and salts, such as oxalate, strontium chloride, silver diamine fluoride, materials based on bioactive glass particle, CPP-ACP-based products, and fluoride-based drugs. Dentinal tubules can also be occluded by protein coagulation using products containing HEMA and glutaraldehyde. However, there are a number of results indicating that the consistency and durability of the effects of these products is questionable. Dentin is also mechanically occluded using dentin adhesive and resin, but when the tooth root surface is exposed due to gingival recession, there are limitations in that access to especially the adjacent surface is difficult and it is difficult to perform a perfect adhesion process without contamination. This treatment method also has a problem in terms of durability. Attempts have also been made to occlude or narrow the dentinal tubules by melting the dentin surface by irradiating exposed dentin with a laser such as Nd:UAG or Er:Cr:YSGG, but there are limitations in that, since only a local portion is melted, the dentinal tubules in the remaining portion are still open, or the applied energy makes the portion around the energy-applied portion more sensitive. Mineral trioxide aggregate (MTA) material can also be used, but it has a long hardening time and stains the teeth black, indicating that the use thereof in the tooth cervical area, which is the main area of hypersensitivity, results in fatal outcome. In addition, this material is difficult to apply to narrow-diameter areas such as dentinal tubules, due to its relatively large particle size. In addition, the method of occluding dentinal tubules using the above material involves the inconvenience of having to visit a dentist for treatment.

Meanwhile, most commercially available products for occluding dentinal tubules have disadvantages in that they do not penetrate deep enough into dentinal tubules, or affect only the dentin surface, or the dentinal tubule-occluding effect thereof does not last because the occlusion material does not remain due to the influence of the changes in the oral environment by the user's food intake or brushing teeth.

Therefore, there is still a need for a novel composition for alleviating dentin hypersensitivity or occluding dentinal tubules, which hat has excellent and lasting dentinal tubule-occluding ability and is easy to apply.

### SUMMARY

An object to be achieved by the present disclosure is to provide a composition that has excellent dentinal tubule-occluding ability and is capable of preventing or alleviating dentin hypersensitivity.

However, objects to be achieved by the present disclosure are not limited to the above-mentioned object, and other objects not mentioned above will be clearly understood by those skilled in the art from the following description.

One embodiment of the present disclosure provides a composition for occluding dentinal tubules including a cement containing 70 wt % or more of at least one of dicalcium silicate (C2S) and tricalcium silicate (C3S), wherein the cement has an average particle diameter (D₅₀) of 2.5 μm or less as measured by a particle size analysis method.

Another embodiment of the present disclosure provides an oral product including the composition for occluding dentinal tubules according to one embodiment of the present disclosure.

Another embodiment of the present disclosure provides a method for preventing or alleviating dentin hypersensitivity, comprising applying a composition for occluding dentinal tubules comprising a cement containing 70 wt % or more of at least one of dicalcium silicate (C2S) and tricalcium silicate (C3S), wherein the cement has an average particle diameter (D50) of 2.5 μm or less as measured by a particle size analysis method.

The composition for occluding dentinal tubules according to one embodiment of the present disclosure may have excellent dentinal tubule-occluding ability.

The composition for occluding dentinal tubules according to one embodiment of the present disclosure may have the effect of preventing and alleviating dentinal hypersensitivity by reducing fluid flow in dentinal tubules and dentin permeability.

The composition for occluding dentinal tubules according to one embodiment of the present disclosure may have a dentinal tubule-occluding effect that can last even when environmental changes such as oral cavity temperature, acidity, pressure, and humidity changes occur due to food intake or teeth brushing.

The oral product according to one embodiment of the present disclosure may have the effect of preventing and alleviating dentin hypersensitivity.

The effects of the present disclosure are not limited to the effects described above, and effects not mentioned above may be clearly understood by those skilled in the art from the present specification and the accompanying drawings.

### BRIEF DESCRIPTION OF THE DRAWINGS

FIG. 1 is a graph showing the particle size distribution of cement powder produced in Production Example 1.

FIG. 2 is a graph showing the particle size distribution of cement powder produced in Production Example 2.

FIG. 3 is a diagram schematically showing a process of preparing a dentin specimen for testing.

FIG. 4 is an SEM image of a longitudinal section of a dentin specimen immersed in PBS medium for 60 days after a composition for occluding dentinal tubules prepared in Example 1 was applied thereto.

FIG. 5 shows SEM images of longitudinal sections of (a) control dentin specimens and (b) dentin specimens to which a composition for occluding dentinal tubules prepared in Example 1 was applied, wherein the specimens were immersed in 1×PBS medium for 1 day, 30 days, 60 days, and 90 days.

FIG. 6 shows SEM images of longitudinal sections of dentin specimens to which a composition of Example 2 was applied, depending on the concentration of PBS medium and the time of immersion in PBS medium.

### DETAILED DESCRIPTION

Throughout the present specification, it is to be understood that when any part is referred to as “including” any component, it does not exclude other components, but may further include other components, unless otherwise specified.

Throughout the present specification, when any member is referred to as being “on” another member, it not only refers to a case where any member is in contact with another member, but also a case where a third member exists between the two members.

Throughout the present specification, “A and/or B” means “A and B” or “A or B”.

Throughout the present specification, “dentin hypersensitivity” refers to symptoms of tooth sensitivity caused by external stimuli such as temperature stimulation or external pressure, or refers to pain caused by tooth sensitivity.

Throughout the present specification, “alleviation” means reducing or relieving symptoms or pain caused by symptoms, and may be understood as a broad concept including amelioration, prevention, treatment, etc.

Throughout the present specification, “dicalcium silicate” or “C2S” refers to 2CaO·SiO₂.

Throughout the present specification, “tricalcium silicate” or “C3S” refers to 3CaO·SiO₂.

Hereinafter, the present disclosure will be described in more detail.

In the present disclosure, to avoid the repeated disclosure, hereinafter the description of composition for occluding dentinal tubules is equally applied to ‘A method for preventing or alleviating dentin hypersensitivity, comprising applying a composition for occluding dentinal tubules’ and ‘an oral product comprising the composition for occluding dentinal tubules’.

### [Composition]

One embodiment of the present disclosure provides a composition for occluding dentinal tubules including a cement containing 70 wt % or more of at least one of dicalcium silicate (C2S) and tricalcium silicate (C3S), wherein the cement has an average particle diameter (D₅₀) of 2.5 μm or less as measured by a particle size analysis method.

The composition for occluding dentinal tubules according to one embodiment of the present disclosure may have excellent dentinal tubule-occluding ability by including a cement containing 70 wt % more of at least one of dicalcium silicate (C2S) and tricalcium silicate (C3S). More specifically, the dicalcium silicate and/or tricalcium silicate component (s) contained in the cement may react with the phosphate component contained in the biological fluid within dentinal tubules to form hydroxyapatite-like crystals within the dentinal tubules, and the formed crystals may effectively seal the inside of the dentinal tubules.

The composition for occluding dentinal tubules according to one embodiment of the present disclosure may have the effect of preventing and alleviating dentin hypersensitivity by sealing the inside of dentinal tubules and reducing fluid flow in the dentinal tubules and dentin permeability.

The composition for occluding dentinal tubules according to one embodiment of the present disclosure can form crystals in the dentinal tubules at a depth of 30 μm or more from the exposed dentin surface. The composition for occluding dentinal tubule according to the present disclosure does not merely seal the area near the dentinal tubule entrances present on the exposed dentin surface, but can also form crystals in dentinal tubules, more specifically, in the dentinal tubules at a depth of 30 μm or more from the exposed dentin surface, and thus the dentinal tubule-occluding effect thereof can last even when environmental changes such as oral cavity temperature, acidity, pressure, and humidity changes occur due to food intake or teeth brushing.

The composition for occluding dentinal tubules according to one embodiment of the present disclosure may be used to alleviate or prevent dentin hypersensitivity.

According to one embodiment of the present disclosure, the composition for occluding dentinal tubules may be applied to teeth by applying the composition so that the composition comes into contact with the tooth surface with exposed dentinal tubules, or by gargling, or by injecting the composition directly into dentinal tubules. Furthermore, the composition for occluding dentinal tubules may be applied to teeth by applying the composition thereto so that the composition comes into contact with the tooth surface with exposed dentinal tubules, and then polishing the tooth surface using a tooth polishing tool such as a toothbrush.

When the composition for occluding dentinal tubules according to one embodiment of the present disclosure is applied to teeth by the above-described method, it is possible to increase the penetration rate of cement particles included in the composition into dentinal tubules, and to improve the dentinal tubule-occluding effect of the composition for occluding dentinal tubules according to the present disclosure by increasing the penetration rate of the cement particles into dentinal tubules, and also to improve the durability of the dentin hypersensitivity prevention and alleviation effects of the composition.

According to one embodiment of the present disclosure, the formulation of the composition for occluding dentinal tubules is not particularly limited, but is preferably, for example, a liquid formulation such as a solution, emulsion, or suspension, or a solid formulation such as powder, or a semi-solid formulation such as a gel, cream, or paste.

According to one embodiment of the present disclosure, the composition for occluding dentinal tubules may include a viscosity modifier to achieve a desired formulation.

According to one embodiment of the present disclosure, the viscosity modifier may be a non-aqueous liquid, an aqueous liquid, or a mixture thereof. The viscosity modifier may be used without particular limitation as long as it facilitates oral application of the composition for occluding dentinal tubules according to one embodiment of the present disclosure, and may be appropriately selected depending on additional components included in the composition.

According to one embodiment of the present disclosure, the non-aqueous liquid may include, for example, at least one selected from among ethanol, propanol, vegetable oil, animal oil, ethylene glycol, propylene glycol, polyethylene glycol, polypropylene glycol, and glycerin, without being limited thereto.

According to one embodiment of the present disclosure, the aqueous liquid may include, for example, at least one selected from among water, purified water, distilled water, PBS, and artificial saliva, without being limited thereto.

### [Cement]

According to one embodiment of the present disclosure, the cement contains 70 wt % or more of at least one of dicalcium silicate (C2S) and tricalcium silicate (C3S). More specifically, the cement may contain 70 wt % or more, 75 wt % or more, 80 wt % or more, or 85 wt % or more of at least one of dicalcium silicate and tricalcium silicate, and the content of dicalcium silicate (C2S) and tricalcium silicate (C3S) may also be 100 wt %.

According to one embodiment of the present disclosure, the cement may be produced by calcining a raw material mixture containing calcium carbonate and silicon dioxide. When the cement is produced by calcining a raw material mixture containing calcium carbonate and silicon dioxide, it is possible to minimize the content of impurities such as ferrite in the produced cement, and it is possible to prevent the tooth surface from being stained when the composition for occluding dentinal tubules is applied to the tooth.

According to one embodiment of the present disclosure, the cement may be obtained by performing a step of calcining the raw material mixture and then rapidly cooling the calcined mixture to a temperature of 15° C. to 25° C. As the cement is produced through the rapid cooling step after calcination, the purity of dicalcium silicate and tricalcium silicate in the produced cement may be high.

According to one embodiment of the present disclosure, the calcining temperature in the production of the cement may be 1,000° C. to 1,500° C., specifically 1,100° C. to 1,500° C., 1,200° C. to 1,500° C., or 1,300° C. to 1,500° C. When the calcining temperature is controlled within the above range, it is possible to control the weight ratio of dicalcium silicate/tricalcium silicate in the produced cement, and prevent tricalcium silicate from being decomposed during the calcining process.

According to one embodiment of the present disclosure, when the cement is produced by calcining the raw material mixture containing calcium carbonate and silicon dioxide, the cement may contain, in addition to the dicalcium silicate and tricalcium silicate components, components such as tricalcium aluminate (3CaO·Al₂O₃), tetracalcium aluminoferrite (4CaO·Al₂O₃·Fe₂O₃), periclase (MgO), lime, etc.

According to one embodiment of the present disclosure, the calcium carbonate may be contained in the raw material mixture in an amount of 50 to 90 parts by weight, 55 to 85 parts by weight, 60 to 90 parts by weight, 60 to 85 parts by weight, or 65 to 85 parts by weight, based on 100 parts by weight of the total weight of the raw material mixture, and the silicon dioxide may be contained in the raw material mixture in an amount of 10 to 40 parts by weight, 10 to 35 parts by weight, 10 to 30 parts by weight, 15 to 30 parts by weight, or 15 to 25 parts by weight, based on 100 parts by weight of the total weight of the raw material mixture. When the weight ratio of calcium carbonate to silicon dioxide contained in the raw material mixture is controlled within the above range, the content of dicalcium silicate and tricalcium silicate in the produced cement may be high, and it is possible to control the dicalcium silicate/tricalcium silicate weight ratio.

According to one embodiment of the present disclosure, the raw material mixture may further contain aluminum oxide in an amount of 3 to 20 parts by weight based on 100 parts by weight of the total weight of the raw material mixture.

According to one embodiment of the present disclosure, the cement has an average particle diameter (D₅₀) of 2.5 μm or less as measured by a particle size analysis method. As the average particle diameter (D₅₀) of the cement is within the range of 2.5 μm or less, the cement particles can penetrate into dentinal tubules having a narrow diameter. As the cement particles penetrate into the dentinal tubules, hydroxyapatite-like crystals can form and grow in the dentinal tubules, and the dentinal tubule-occluding effect can last even when environmental changes such as oral cavity temperature, acidity, pressure, and humidity changes occur due to food intake or teeth brushing. More specifically, as hydroxyapatite-like crystals form and grow in the dentinal tubules at a depth of 50 μm or more from the exposed dentin surface, the dentinal tubule-occluding effect can last even when environmental changes such as oral cavity temperature, acidity, pressure, and humidity changes occur due to food intake or teeth brushing.

According to one embodiment of the present disclosure, the cement may have an average particle diameter (D₅₀) of 2.5 μm or less, 2.0 μm or less, 1.5 μm or less, 1.0 μm or less, 0.8 μm or less, or 0.5 μm or less, as measured by a particle size analysis method. When the cement has an average particle size within the above range, it is possible to increase the amount of dicalcium silicate and tricalcium silicate components penetrating into dentinal tubules, thereby increasing the lasting time of the dentinal tubule-occluding ability of the composition for occluding dentinal tubules and reducing the time taken until the dentinal tubule-occluding effect appears.

More specifically, the cement may have an average particle diameter (D₅₀) of 0.001 μm or more, 0.005 μm or more, 0.01 μm or more, or 0.05 μm or more, and 2.5 μm or less, 2.0 μm or less, 1.5 μm or less, 1.0 μm or less, 0.8 μm or less, or 0.5 μm or less, as measured by a particle size analysis method. When the cement has an average particle diameter satisfying the upper limit of the above range, it is possible to increase the amount of dicalcium silicate and tricalcium silicate components penetrating into dentinal tubules, thereby increasing the lasting time of the dentinal tubule-occluding ability of the composition for occluding dentinal tubules and reducing the time taken until the dentinal tubule-occluding effect appears. In addition, when the cement has an average particle diameter satisfying the lower limit of the above range, a grinding process of grinding the cement, which is a bulk material, may be time- and cost-effective.

### [Other Components]

The composition for occluding dentinal tubules according to one embodiment of the present disclosure may further include a phosphate source. Specifically, the phosphate source may be at least one of saliva, artificial saliva, and phosphate buffered saline (PBS), without being limited thereto. As the composition for occluding dentinal tubules further includes the phosphate source, the concentration of phosphate, which is a reactant required to form hydroxyapatite-like crystals, may further increase near the cement containing the dicalcium silicate and/or tricalcium silicate.

According to one embodiment of the present disclosure, the phosphate buffered saline may have a phosphate concentration of 0.01 M or more. More specifically, the phosphate concentration of the phosphate buffered saline may be 0.01 M to 1 M, 0.02 M to 1.00 M, 0.05 M to 1.00 M, or 0.1 M to 1 M. As the phosphate concentration of the phosphate-buffered saline contained in the composition for occluding dentinal tubules according to one embodiment of the present disclosure is within the above range, it is possible to promote the formation and growth of hydroxyapatite-like crystals in dentinal tubules. Therefore, it is possible to increase the lasting time of the dentinal tubule-occluding effect of the composition and reduce the time taken until the dentinal tubule-occluding effect appears.

The composition for occluding dentinal tubules according to one embodiment of the present disclosure may further include conventional components, such as a wetting agent, an abrasive, an active pharmaceutical ingredient, a sweetener, a pH adjuster, a binder, a whitening agent, etc., depending on the formulation and intended use of a product including the composition.

According to one embodiment of the present disclosure, the wetting agent may be selected from the group consisting of concentrated glycerin, glycerin, sorbitol aqueous solution, amorphous sorbitol aqueous solution, polyethylene glycols, propylene glycol, and mixtures thereof.

According to one embodiment of the present disclosure, the abrasive may be selected from the group consisting of precipitated silica, silica gel, zirconium silicate, calcium monohydrogen phosphate, anhydrous calcium monohydrogen phosphate, hydrous alumina, precipitated calcium carbonate, ground calcium carbonate, calcium pyrophosphate, insoluble metaphosphate, aluminum silicate, and mixtures thereof.

According to one embodiment of the present disclosure, the active pharmaceutical ingredient may be selected from the group consisting of sodium fluoride, sodium monofluorophosphate, stannous fluoride, chlorhexidine, allantoin chlorohydroxyaluminate, aminocaproic acid, zinc chloride, pyridoxine hydrochloride, tocopherol acetate, enzymes, and mixtures thereof.

According to one embodiment of the present disclosure, the sweetener may be selected from the group consisting of saccharin, xylitol, erythritol, aspartame, and mixtures thereof.

According to one embodiment of the present disclosure, the pH adjuster may be selected from the group consisting of sodium phosphate, disodium phosphate, citric acid, triethanolamine, and mixtures thereof.

According to one embodiment of the present disclosure, the binder may be selected from the group consisting of carrageenan, xanthan gum, sodium carboxymethyl cellulose, carboxyvinyl polymers, sodium alginate, laponite, and mixtures thereof.

According to one embodiment of the present disclosure, the whitening agent may be selected from the group consisting of peroxides, whitening functional fruit and plant extracts, titanium oxide, and mixtures thereof.

Another embodiment of the present disclosure provides an oral product including the composition for occluding dentinal tubules according to one embodiment of the present disclosure.

According to one embodiment of the present disclosure, the oral product may be used to alleviate or prevent dentin hypersensitivity.

The oral product according to one embodiment of the present disclosure may have the effect of preventing and alleviating dentin hypersensitivity when used by including the composition for occluding dentinal tubules according to one of the present disclosure.

According to one embodiment of the present disclosure, the oral product may be provided in at least one form selected from among toothpastes, oral sprays, mouthwashes, mouth fresheners, tooth patches, desensitizers, and tooth remineralizers, but is not limited thereto and may be in any form that allows the composition for occluding dentinal tubules to come into contact the tooth surface with exposed dentinal tubules.

Hereinafter, the present disclosure will be described in detail with reference to examples. However, the examples according to the present disclosure may be modified into various different forms, and the scope of the present disclosure is not interpreted as being limited to the examples described below. The examples of the present specification are provided to more completely explain the present disclosure to those skilled in the art.

### Production Example 1: Production of Cement Containing Dicalcium Silicate and Tricalcium Silicate

600 g of calcium carbonate (CaCO₃), 170 g of silicon dioxide (SiO₂), and 30 g of aluminum oxide (Al₂O₃), which were dried at 100° C. for 24 hours or more, were weighed as raw materials, placed in a ceramic bowl, and mixed evenly to obtain a raw material mixture. The raw material mixture was placed in a platinum crucible and calcined in a furnace at a temperature of 1,450° C. for about 6 hours. Immediately after the calcining process, the resulting material was taken out of the furnace and rapidly cooled to 25° C. within 10 minutes using a cooling fan in the air, thereby synthesizing cement.

Then, the cement was subjected to a first grinding process using a disk mill (KM Tech) at 300 rpm under dry conditions, and subjected to a second grinding process using a ball mill (BML-2, DAITHAN SCIENTIFIC GROUP) at 200 rpm for 48 hours.

After grinding, the cement powder was analyzed by a particle size analyzer (Mastersizer S, Malvern).

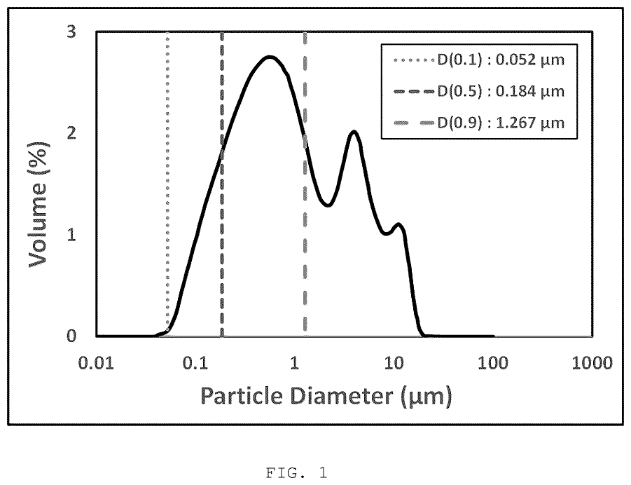

FIG. 1 is a graph showing the particle size distribution of the cement powder produced in Production Example 1. Referring to FIG. 1, the cement powder produced in Production Example 1 had a D₁₀ (D_{0.1}) of 0.052 μm, a D₉₀ (D_{0.9}) of 1.267 μm, and an average particle diameter (D₅₀) of 0.184 μm.

Table 1 below shows the components and component contents of the cement produced in Production Example 1.

TABLE 1

| Cement | | Content |
|---|---|---|
| (n = 12) | Component | (wt %) |
| Production | Dicalcium silicate (2CaO•SiO₂; | 14.91 |
| Example 1 | C2S) | |
| (n = 12) | Tricalcium silicate (3CaO•SiO₂; | 74.21 |
| | C3S) | |
| | Other components: | 10.88 |
| | tricalcium aluminate (3CaO•Al₂O₃; | |
| | C3A), tetracalcium aluminoferrite | |
| | (4CaO•Al₂O₃•Fe₂O₃; C4AF), periclase | |
| | (MgO), and lime | |

### Production Example 2: Production of Cement Containing Tricalcium Silicate

300 g of calcium carbonate (CaCO₃) and 60 g of silicon dioxide (SiO₂), which were dried at 100° C. for 24 hours or more, were weighed as raw materials, placed in a ceramic bowl, and mixed evenly to obtain a raw material mixture. The raw material mixture was placed in a platinum crucible and calcined in a furnace at a temperature of 1,450° C. for about 6 hours. Immediately after the calcining process, the resulting material was taken out of the furnace and rapidly cooled to 25° C. within 10 minutes using a cooling fan in the air, thereby synthesizing cement.

Then, the cement was subjected to a first grinding process using a disk mill (KM Tech) at 300 rpm under dry conditions, and subjected to a second grinding process using a ball mill (BML-2, DAITHAN SCIENTIFIC GROUP) at 200 rpm for 48 hours.

After grinding, the cement powder containing tricalcium silicate was analyzed by a particle size analyzer (Mastersizer S, Malvern).

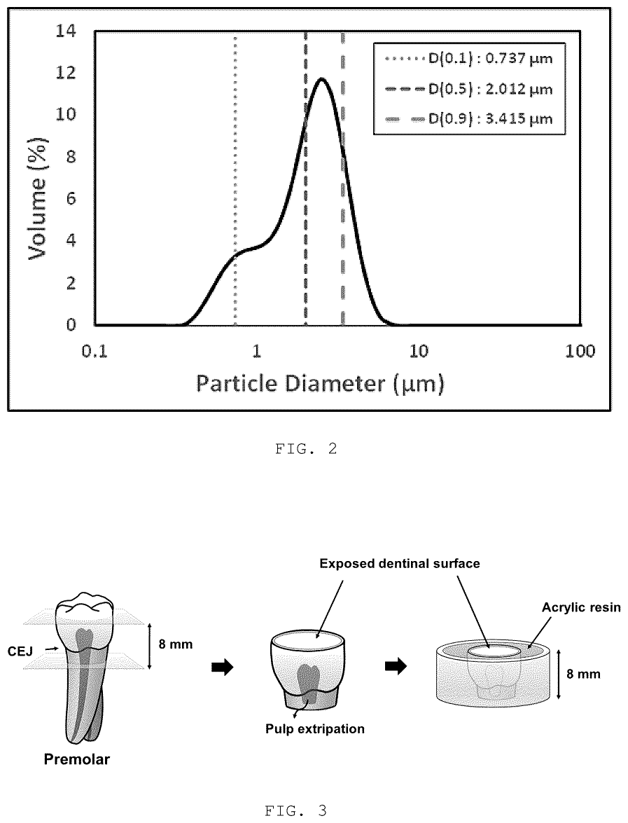

FIG. 2 is a graph showing the particle size distribution of cement powder produced in Production Example 2. Referring to FIG. 2, the cement powder produced in Production Example 2 had a D₁₀ (D_{0.1}) of 0.737 μm, a D₉₀ (D_{0.9}) of 3.415 μm μm, and an average particle diameter (D₅₀) of 2.012 μm.

### Example 1: Preparation of Composition for Occluding Dentinal Tubules

0.5 g of the cement produced according to Production Example 1 was added to and mixed with 5 ml of distilled water, thereby preparing a composition for occluding dentinal tubules.

### Example 2: Preparation of Composition for Occluding Dentinal Tubules

0.5 g of the cement produced according to Production Example 2 was added to and mixed with 5 ml of distilled water, thereby preparing a composition for occluding dentinal tubules.

### Test Example 1: Test for Crystal Formation in Dentinal Tubules

### 1-1. Preparation of Dentin Specimens

A total of 24 human premolars with intact coronal and root surfaces, recently extracted for orthodontic treatment, were prepared. The extracted premolars were stored in 0.1% thymol solution to inhibit microbial growth for more than 3 months before use. Foreign substances on all the tooth surfaces were removed with a periodontal curette, and whether there were fracture lines in the premolars was examined under a microscope (200×) (Carl Zeiss Surgical GmbH, Oberkochen, Germany).

FIG. 3 is a diagram schematically showing a process of preparing a dentin specimen for testing. The specimen preparation process will be explained with reference to FIG. 3. The extracted premolar was cut at 4 mm above and below from the cemento-enamel junction(CEJ) using a low-speed diamond saw (Isomet™, Buehler, Lake Bluff, IL, USA) under constant water-cooling, thus preparing a specimen with a total length of 8 mm. Flat and fresh dentin was then exposed on the occlusal side surface without perforation at the pulp horn area. The remaining pulp tissue was removed carefully with small forceps without touching the inner part of the pulpal space. Then, the sectioned tooth was mounted in the ring-shaped acrylic mold with self-cured acrylic resin (Bosworth Fastray, Keystone Industries GmbH, Singen, Germany). The exposed upper dentin surface of each specimen was treated with 17% ethylenediaminetetraacetic acid (EDTA) for 1 minute, and then treated with 2.5 mL of 5.25% sodium hypochlorite to open dentinal tubules and remove the smear layer on the exposed dentin surface, followed by rinsing with 10 mL of distilled water twice.

### 1-2. Method of Applying Composition for Occluding Dentinal Tubules to Dentin Specimen

The composition for occluding dentinal tubules prepared in Example 1 was applied to the exposed dentin surface of the specimen prepared in 1-1 above. Then, tooth brushing motion was performed on the dentin surface to which the composition had been applied, according to ISO 11609 standards for dentin abrasion test. Specifically, a total of 10,000 repeated strokes (1 stroke/sec) were applied using a toothbrush onto each specimen under a 150 g-load continuously being touched among the composition prepared in Example 1 and the exposed dentin surface of the specimen.

### 1-3. Confirmation of Crystal Formation in Dentinal Tubules

The specimens to which the composition for occluding dentinal tubules had been applied as described in 1-2 above were randomly divided into four subgroups according to the period of immersion in PBS (D8662, Sigma-Aldrich, St. Louis, MO, USA) medium for 1, 30, 60, and 90 days each at 37° C. (n=3). The PBS solution was replaced every 7 days. The composition of 1×PBS used was CaCl₂·2H₂O 0.133 g/L, MgCl₂·6H₂O 0.1 g/L, KCl 0.2 g/L, KH₂PO₄ 0.2 g/L, NaCl 8.0 g/L, Na₂HPO₄ (anhydrous) 1.15 g/L, and the pH was 7.4.

To assess crystal formation in dentinal tubules, the dentin specimens to which the composition for occluding dentinal tubules had been applied were longitudinally sectioned, and six sectioned surfaces in each group were examined. All specimens were mounted on aluminum stubs and sputter-coated with a 30-nm layer of gold, and then whether crystals were formed in the dentinal tubules was examined using field emission scanning electron microscopy (FE-SEM, Apreo S; Thermo Fisher SCIENTIFIC, Waltham, MA, USA).

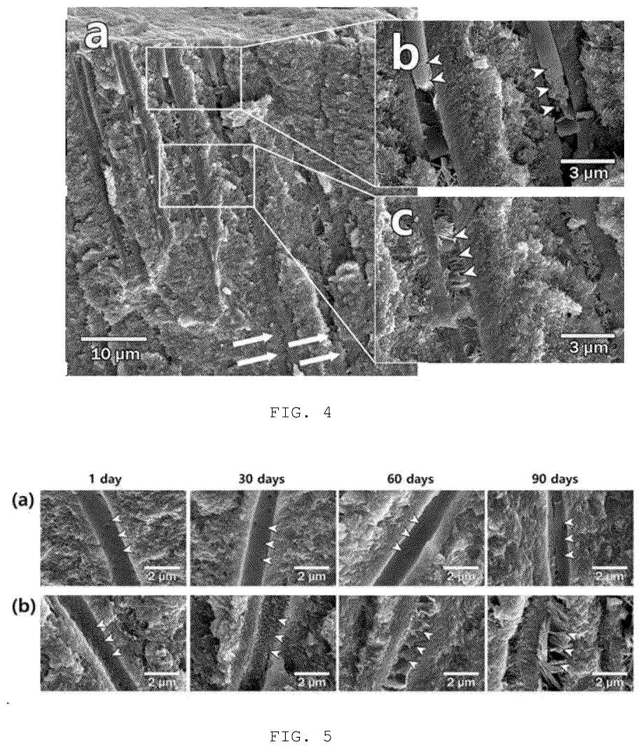

FIG. 4 is an SEM image of a longitudinal section of the dentin specimen immersed in PBS medium for 60 days after the composition for occluding dentinal tubules prepared in Example 1 was applied thereto.

Specifically, part b of FIG. 4 shows an SEM image of a plug-shaped precipitate formed in dentinal tubules near the exposed dentin surface.

Part c FIG. 4 shows an SEM image of plate-shaped crystals formed in dentinal tubules.

Referring to FIG. 4, it could be confirmed that, in the dentin specimen to which the composition for occluding dentinal tubules prepared in Example 1 was applied, there were plug-shaped precipitates near the exposed dentin surface, and plate-shaped crystals were formed in the dentinal tubules at a depth of about 50 μm or more from the exposed dentin surface.

1-4. Confirmation of Crystal Growth in Dentinal Tubules Over Time after Application of Composition for Occluding Dentinal Tubules and Analysis of Crystal Components in Dentinal Tubules

For the dentin specimens immersed in PBS medium for 1 day, 30 days, 60 days, and 90 days after the composition for occluding dentinal tubules prepared in Example 1 was applied, whether crystals were formed in the dentinal tubules was examined in the same manner as in 1-3 above using field emission scanning electron microscopy (FE-SEM, Apreo S; Thermo Fisher SCIENTIFIC, Waltham, MA, USA).

In addition, as a control group, dentin specimens were prepared in the same manner as in 1-2 above, except that plain water was used instead of the composition for occluding dentinal tubules prepared in Example 1.

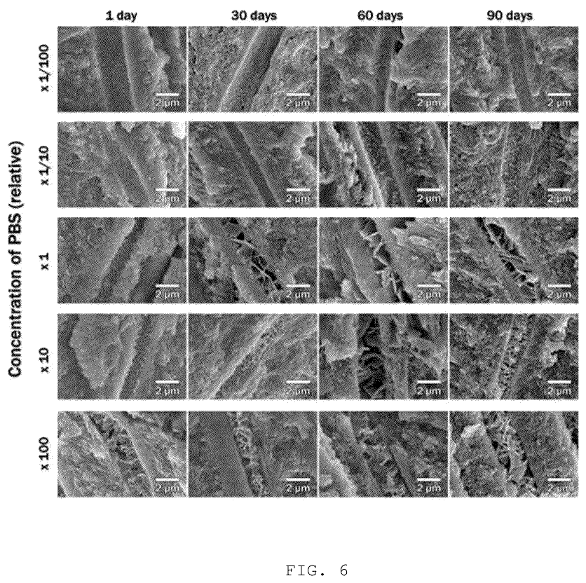

FIG. 5 shows SEM images of longitudinal sections of (a) control dentin specimens and (b) dentin specimens to which a composition for occluding dentinal tubules prepared in Example 1 was applied, wherein the specimens were immersed in 1×PBS medium for 1 day, 30 days, 60 days, and 90 days.

Referring to part (a) of FIG. 5, it was confirmed that, in the control specimens to which the composition for occluding dentinal tubules prepared in Example 1 was not applied, no crystals were formed in the dentinal tubules in all the specimens regardless of the time of immersion in PBS medium.

Referring to part (b) of FIG. 5, it was confirmed that, in the specimens to which the composition for occluding dentinal tubules prepared in Example 1 was applied, denser crystals were formed in the dentinal tubules as the time of immersion in PBS medium increased. Specifically, it could be confirmed that, in the specimen immersed for 1 day, the inner surface of the dentinal tubules became rough, and in the specimen immersed for 60 days, plate-shaped crystals were formed in the dentinal tubules. It was confirmed that, as the period of immersion in the PBS medium got longer, the plate-shaped crystals were linked together to form high-density crystals.

To analyze the components of the crystals formed in the dentinal tubules, the specimens to which the composition for occluding dentinal tubules prepared in Example 1 had been applied was examined by energy dispersive spectroscopy (EDS, XFlash 6160, Bruker, Germany).

Table 2 shows the Ca/P ratio of crystals formed in the dentinal tubules of the dentin specimens immersed in 1×PBS medium for 1 day, 30 days, 60 days, and 90 days after the composition for occluding dentinal tubules prepared in Example 1 was applied.

TABLE 2

Time of immersion in 1X PBS medium

| | 1 day | 30 days | 60 days | 90 days |
|---|---|---|---|---|
| Ca/P | 1.57 | 1.59 | 1.64 | 1.68 |

The Ca/P ratio of crystals formed in the dentinal tubules in the dentin specimen immersed in PBS medium for 1 day after the composition for occluding dentinal tubules prepared in Example 1 was applied thereto was 1.57. The Ca/P ratio of the crystals increased to 1.68 in the specimen immersed in PBS medium for 90 days. The Ca/P ratio of hydroxyapatite (HAp, Ca₁₀(PO₄)₆(OH)₂) is known to be 1.67.

These results indicate that the crystals formed from the C2S and/or C3S component(s) contained in the composition for occluding dentinal tubules prepared in Example 1 grew into crystals of components chemically similar to those of hydroxyapatite over time in PBS medium.

### Test Example 2: Test for Crystal Formation in Dentinal Tubules Depending on PBS Concentration

Dentin specimens for testing were prepared in the same manner as described in 1-1 above.

The method of applying the composition for occluding dentinal tubules to the dentin specimens was performed in the same manner as described in 1-2 above, except that the composition prepared in Example 2 was used instead of the composition prepared in Example 1.

Each specimen to which the composition for occluding dentinal tubules was applied thereto as described above was immersed in 1×PBS (D8662, Sigma-Aldrich, St. Louis, MO, USA) medium, 1/100×PBS medium, 1/10×PBS medium, 10×PBS medium, and 100×PBS medium for 1 day, 30 days, 60 days, and 90 days each at 37° C. The specimens were randomly divided into four subgroups according to the immersion period and the concentration of PBS medium (n=3). The PBS solution was replaced every 7 days. The composition of 1×PBS as a reference was CaCl₂·2H₂O 0.133 g/L, MgCl₂·6H₂O 0.1 g/L, KCl 0.2 g/L, KH₂PO₄ 0.2 g/L, NaCl 8.0 g/L, Na₂HPO₄ (anhydrous) 1.15 g/L, and the pH was 7.4.

To assess crystal formation in the dentinal tubules, the dentin specimens were longitudinally sectioned, and six sectioned surfaces in each group were examined. All specimens were mounted on aluminum stubs and sputter-coated with a 30-nm layer of gold, and then whether crystals were formed in the dentinal tubules was examined using field emission scanning electron microscopy (FE-SEM, Apreo S; Thermo Fisher SCIENTIFIC, Waltham, MA, USA).

FIG. 6 shows SEM images of longitudinal sections of the dentin specimens to which the composition of Example 2 was applied, depending on the concentration of the PBS medium and the time of immersion in the PBS medium.

Referring to FIG. 6, it was confirmed that plate-shaped crystals were formed in the dentinal tubules of the dentin specimen immersed in 1×PBS medium for 30 days after the composition of Example 2 was applied thereto, and that, as the immersion time increased, plate-shaped crystals grew to form dense crystals.

In addition, it was confirmed that the dentin specimen immersed in 10×PBS medium or 100×PBS medium after the composition of Example 2 was applied was applied thereto showed faster crystal formation and crystal growth rates than those in 1×PBS medium. Specifically, it could be confirmed that, in the dentin specimen immersed in 100×PBS medium for 1 day after the composition of Example 2 was applied thereto, crystals were formed in the dentinal tubules, and it was confirmed that, in the dentin specimen immersed in 100×PBS medium for 30 days after the composition of Example 2 was applied thereto, plate-shaped crystals were linked together to form high-density crystals.

Although the present disclosure has been described above by way of limited embodiments, the present disclosure is not limited thereto. It should be understood that the present disclosure can be variously changed and modified by those skilled in the art without departing from the technical sprit of the present disclosure and the range of equivalents to the appended claims.

What is claimed is:

1. A composition for occluding dentinal tubules comprising a cement containing 70 wt % or more of at least one of dicalcium silicate (C2S) and tricalcium silicate (C3S), wherein the cement has an average particle diameter (D₅₀) of 2.5 μm or less as measured by a particle size analysis method.

2. The composition according to claim 1, further comprising a phosphate source.

3. The composition according to claim 2, wherein the phosphate source is at least one of saliva, artificial saliva, and phosphate buffered saline (PBS).

4. The composition according to claim 2, wherein the phosphate source is phosphate buffered saline, and the phosphate buffered saline has a phosphate concentration of 0.01 M or more.

5. The composition according to claim 1, wherein the cement has an average particle diameter (D₅₀) of 2.0 μm or less as measured by a particle size analysis method.

6. The composition according to claim 1, which forms crystals in dentinal tubules at a depth of 30 μm or more from an exposed dentin surface.

7. The composition according to claim 1, wherein the cement is produced by calcining a raw material mixture containing calcium carbonate and silicon dioxide.

8. The composition according to claim 7, wherein the calcining is performed at a temperature of 1,000° C. to 1,500° C.

9. An oral product comprising the composition for occluding dentinal tubules according to claim 1.

10. The oral product according to claim 9, which is provided in at least one form selected from among toothpastes, oral sprays, mouthwashes, mouth fresheners, tooth patches, desensitizers, and tooth remineralizers.

11. A method for preventing or alleviating dentin hypersensitivity, comprising applying a composition for occluding dentinal tubules comprising a cement containing 70 wt % or more of at least one of dicalcium silicate (C2S) and tricalcium silicate (C3S), wherein the cement has an average particle diameter (D50) of 2.5 μm or less as measured by a particle size analysis method.

12. The method according to claim 11, wherein the composition further comprising a phosphate source.

13. The method according to claim 12, wherein the phosphate source is at least one of saliva, artificial saliva, and phosphate buffered saline (PBS).

14. The method according to claim 12, wherein the phosphate source is phosphate buffered saline, and the phosphate buffered saline has a phosphate concentration of 0.01 M or more.

15. The method according to claim 11, wherein the cement has an average particle diameter (D₅₀) of 2.0 μm or less as measured by a particle size analysis method.

16. The method according to claim 11, which forms crystals in dentinal tubules at a depth of 30 μm or more from an exposed dentin surface.

17. The method according to claim 11, wherein the cement is produced by calcining a raw material mixture containing calcium carbonate and silicon dioxide.

18. The method according to claim 17, wherein the calcining is performed at a temperature of 1,000° C. to 1,500° C.

19. The method according to claim 11, wherein the composition is applied to teeth by applying the composition so that the composition comes into contact with the tooth surface with exposed dentinal tubules, or by gargling, or by injecting the composition directly into dentinal tubules.
